(19) United States
(12) Patent Application Publication
(10) Pub. No.: US 2025/0017475 A1
(43) Pub. Date: Jan. 16, 2025

(54) SYSTEMS AND METHODS FOR ANALYZING BLOOD FLOW IN A SUBJECT

(71) Applicant: BSC INNOVATIONS, LLC, Windermere, FL (US)

(72) Inventor: Nathan Gold, Windermere, FL (US)

(73) Assignee: BSC INNOVATIONS, LLC, Windermere, FL (US)

(21) Appl. No.: 18/710,409

(22) PCT Filed: Nov. 23, 2022
(86) PCT No.: PCT/US2022/050872

§ 371 Date: May 15, 2024

Related U.S. Application Data
(60) Provisional application No. 63/282,232, filed on Nov. 23, 2021.

(51) Int. Cl. A61B 5/021; A61B 5/00; A61B 5/026; G06T 7/00

(52) U.S. Cl. CPC A61B 5/021; A61B 5/0261; A61B 5/443; A61B 5/725; G06T 7/0016; G06T 2207/10004; G06T 2207/10016; G06T 2207/20081; G06T 2207/20084; G06T 2207/30088; G06T 2207/30201

(57) ABSTRACT

A method for analyzing blood flow in a subject includes generating image data of an area of skin of the subject. The image data is reproducible as one or more images of the area of skin of the subject and/or one or more videos of the area of skin of the subject. The method further includes analyzing at least a portion of the image data to determine a concentration of one or more chromophores within the area of skin of the subject. The method further includes determining, based at least in part on the concentration of the one or more chromophores, a value of at least one metric associated with blood flow of the subject.

### CROSS-REFERENCE TO RELATED APPLICATIONS

This application claims priority to and the benefit of U.S. Provisional Patent Application No. 63/282,232, filed Nov. 23, 2021, which is hereby incorporated by reference.

### TECHNICAL FIELD

The present disclosure relates to systems and methods for analyzing blood flow in a subject, and more particularly, to systems and methods for determining blood pressure from chromophore concentration.

### BACKGROUND

Elevated blood pressure is a leading contributor to cardiovascular disease. Therefore, measuring blood pressure accurately and timely is important for monitoring and/or preventing cardiovascular disease. However, conventional brachial artery blood pressure measurement devices are inconvenient and uncomfortable because it relies on inflatable cuff-based technology. Further, it is very inconvenient to carry the conventional blood pressure measurement device when a person needs to measure blood pressure while travelling or during outdoor activities. Given the high number of people who need to measure blood pressure periodically or frequently, there is an unmet need and large demand to develop a portable, easy, inexpensive, and widely available method for measuring blood pressure.

### SUMMARY

A method for analyzing blood flow in a subject includes generating image data of an area of skin of the subject. The image data is reproducible as one or more images of the area of skin of the subject and/or one or more videos of the area of skin of the subject. The method further includes analyzing at least a portion of the image data to determine a concentration of one or more chromophores within the area of skin of the subject. The method further includes determining, based at least in part on the concentration of the one or more chromophores, a value of at least one metric associated with blood flow of the subject.

A method of training one or more machine learning algorithms includes generating a skin reflectance model describing a spectral reflectance of skin tissue. The method further includes generating a plurality of training data points using the skin reflectance model, each of the plurality of training data points including (i) a pixel color value and (ii) a respective concentration of one or more chromophores corresponding to the pixel color value. The method further includes training the one or more machine learning algorithms with the training data such that the one or more machine learning algorithms are trained to determine a concentration of one or more chromophores in an area of skin of the subject based at least in part on image data associated with the area of skin of a subject.

The above summary is not intended to represent each implementation or every aspect of the present disclosure. Additional features and benefits of the present disclosure are apparent from the detailed description and figures set forth below.

### BRIEF DESCRIPTION OF THE FIGURES

FIG. 1A illustrates a flowchart of a method for analyzing blood flow in a subject, according to aspects of the present disclosure;

FIG. 1B illustrates a flowchart of sub-steps of the method of FIG. 1A, according to aspects of the present disclosure.

FIG. 2 illustrates a flowchart of a method for training a machine learning algorithm to determine chromophore concentration based on image data, according to aspects of the present disclosure;

FIG. 3 illustrates a flowchart of a method for training a machine learning algorithm to determine blood pressure based on chromophore concentration, according to aspects of the present disclosure; and

FIG. 4 is a block diagram of a system for implementing any of the methods of FIGS. 1A, 1i, 2, and 3, according to aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DESCRIPTION OF THE INVENTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

The appearance of skin is determined by the interaction of photons with molecules called chromophores contained in the many layers of skin tissue. Principal amongst the chromophores are melanin and hemoglobin which exist in different concentrations in the layers of the skin. The concentration of these molecules at various layers largely controls the color of human skin tissue. Hemodynamics caused by the motion of blood through the cardiac cycle moves hemoglobin throughout the vasculature. The movement of hemoglobin causes minute and short term variation in the color of human skin which cannot be observed through traditional videographic analysis or color vision. Disclosed herein are systems and methods for analyzing hemodynamics in a subject (e.g., a human subject) using image data.

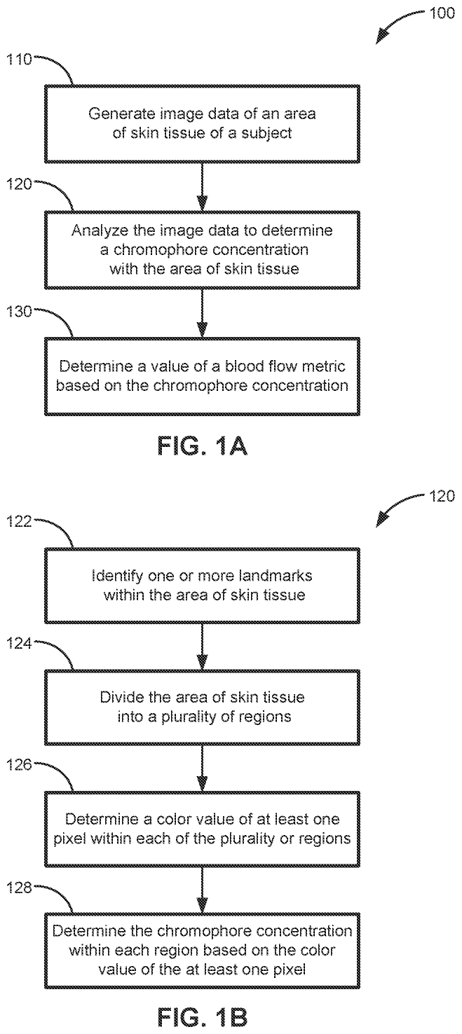

FIG. 1A is a flowchart of a method 100 for analyzing blood flow in a subject. At step 110 of method 100, image data of an area of skin tissue of a subject is generated and/or received. In some implementations, the area of skin tissue includes the subject's face (wholly or partially). In other implementations, the area of skin tissue includes a different portion of the subject's body, such as the subject's arm, leg, chest, back, shoulder, hand, foot, etc.

The image data can be generated using any suitable image capture device(s) with an image sensor (e.g., a CMOS image sensor, a CCD image sensor, etc.). For example, the image capture device can include a digital camera, a digital video camera, or any other suitable device. The image data is reproducible as one or more images and/or one or more videos of the area of skin of the subject. In some implementations, the images data is generated using a digital video camera that records the area of skin over a period of time. The image data is thus representative of the area of skin tissue over time, and can be divided into a plurality of frames (e.g., frames of a video), where each frame is reproducible as an image of the area of skin at different distinct points in time during the period of time. Generally, the image data will be generated while the area of skin tissue of the subject is being illuminated by one or more illumination sources (e.g., light sources), such as an overhead light, a fluorescent light, etc.

At step 120 of method 100, the image data is analyzed to determine a concentration of one or more chromophores within the area of skin tissue (generally referred to herein as a chromophore concentration). In some implementations, the concentration of the one or more chromophores is the volume of the chromophores within the area of skin tissue (or a portion of the area of skin tissue) as a percentage of the overall volume of tissue. The chromophores can include hemoglobin and/or melanin, but can additionally or alternatively include keratin (also referred to as carotene), pheomelanin, bilirubin, fat, and others.

In some implementations, the chromophore concentration determined at step 120 is the concentration of hemoglobin in the area of skin tissue (or in a specific layer or layers of the area of skin tissue). In some implementations, the chromophore concentration determined at step 120 is the concentration of hemoglobin in the area of skin tissue (or in a specific layer or layers of the area of skin tissue) and the concentration of melanin in the area of skin tissue (or in a specific layer or layers of the area of skin tissue). In some implementations, the chromophore concentration determined at step 120 is a single concentration of hemoglobin and melanin in the area of skin tissue (or in a specific layer or layers of the area of skin tissue). In some implementations, the determination of the chromophore concentration is based at least in part on one or more characteristics of the illumination sources being used to illuminate the area of skin tissue (e.g., the identity of the illumination sources). Generally, the chromophore concentration determined at step 120 can include any number of distinct chromophore concentration measurements.

At step 130 of method 100, the value of at least one metric associated with blood flow of the subject is determined based at least in part on the chromophore concentration in the area of skin tissue of the subject. The blood flow metric can be blood pressure, systolic blood pressure, diastolic blood pressure, mean arterial pressure, etc. In some implementations, the subject's blood pressure (or other blood flow metric) is determined based on a single chromophore concentration measurement. In other implementations, the subject's blood pressure (or other blood flow metric) is determined based on a plurality of chromophore concentration measurements across a period of time, as discussed herein. In some implementations, a number of chromophore concentration measurements amounting to about 30 seconds of image data is required to generate a single blood pressure measurement. In some implementations, a plurality of blood pressure measurements is generated from the chromophore concentration measurements (e.g., from the temporal chromophore signal), such that a time-varying blood pressure signal can be formed. Systolic, diastolic, and mean arterial blood pressure can be obtained from this time-varying blood pressure signal.

FIG. 1B is a flow chart that shows the sub-steps of step 120 of method 100. At sub-step 122 of step 120, the image data is analyzed to identify one or more landmarks within the area of skin tissue. For example, if the area of skin tissue is the subject's face, sub-step 122 can include identifying landmarks such as the subject's nose, the subject's mouth, the subject's eyes, the subject's ears, etc. Because the area of skin tissue can move as the image data is being generated (e.g., the subject may move their face as the video is recorded), the area of skin tissue can be identified based on its location relative to these landmarks, instead of its location within the frame of the image data.

Sub-step 124 of step 120 includes dividing the area of skin tissue into a plurality of regions, based at least in part on the identified landmarks. In some implementations, the area of skin tissue is divided into individual groups of pixels. For example, sub-step 124 can include dividing the area of skin tissue into 9×9 groups of pixels. In other implementations, the area of skin tissue is divided on a more macro level. For example, sub-step 124 can include dividing the area of skin tissue in half (e.g., left half of face vs. right half of face, upper half of face vs. lower half of face, etc.), dividing the area of skin tissue into macro portions (e.g., left cheek region, right cheek region, chin region, forehead region, etc.), and other divisions.

Sub-step 126 of step 120 includes determining the color value of at least one pixel within each of the plurality of regions. In implementations where each of the plurality of regions includes a group of pixels, sub-step 126 can include determining the average color value of the pixels within the group of pixels. For example, if at sub-step 124 the area of skin tissue is divided into 9×9 groups of pixels, sub-step 126 can include, for each 9×9 group of pixels, determining the average color value of the group of 9×9 pixels, which is generally the arithmetic average of the 81 pixels forming the 9×9 group of pixels. In some implementations, the color value of a pixel includes a red value, a green value, and a blue value. The average color value of a region can thus include separate averages of the red value, the green value, and the blue value of all of the pixels that forming the region.

Sub-step 128 of step 120 includes determining the chromophore concentration of each region of the area of skin tissue based at least in part on the color value of the at least one pixel in the respective region of the area of skin tissue. Thus, for implementations where the area of skin tissue is the subject's face and the regions are 9×9 groups of pixels, sub-step 128 can include determining the concentration of one or more chromophores for each group of 9×9 pixels of the subject's face in the image data.

In implementations where the image data is generated by a digital video camera and includes a plurality of frames, the chromophore concentration for each region of the area of skin tissue can be determined for each frame. Thus, in these implementations, step 120 can include determining a temporal chromophore for the area of skin that represents a spatial variation in the chromophore concentration within the area of skin tissue (e.g., variation in chromophore concentration across the different regions of the area of skin) and a temporal variation in chromophore concentration within the area of skin tissue across the period of time. In some implementations, a single blood pressure (or other blood flow metric) value for the subject is determined based at least in part on the temporal chromophore signal. In other implementations, a plurality of blood pressure (or other blood flow metric) values for the subject is determined based at least in part on the temporal chromophore signal, which themselves can form a time-varying blood pressure signal. In some implementations, a number of chromophore concentration measurements corresponding to about 30 seconds of image data is used to obtain a single blood pressure (or other blood flow metric) value.

In some implementations, one or more filtering operations can be applied to the temporal chromophore signal to remove the influence of the subject's cardiac cycle on the temporal chromophore signal. For example, the filtering operations can filter out variations having a frequency corresponding to the frequency of the subject's cardiac cycle, which in some implementations can be between about 0.01 Hz and about 5.0 Hz. The filtering operations can include, for example, a Butterworth filter, an elliptical filter, a band-pass filter, other filters, combinations of filters, etc. In some implementations, model decomposition techniques can be applied to the temporal chromophore signal to smooth and/or de-noise the temporal chromophore signal. These model decomposition techniques can include empirical model decomposition, variational model decomposition, etc.

In some implementations, all or part of steps 110, 120, and 130, and/or sub-steps 122, 124, 126, and 128 can be performed by one or more trained machine learning algorithms. For example, in some implementations, analyzing the image data at step 120 includes inputting the image data into one or more machine learning algorithms that have been trained to output an indication of the chromophore concentration in the area of tissue of the subject based at least in part on the image data.

In these implementations, the one or more machine learning algorithms are configured to perform one or more of the sub-steps of step 120. For example, in some implementations, a first set of one or more machine learning algorithms operate on the image data to identify landmarks within the area of skin tissue (sub-step 122), divide the area of skin tissue into a plurality of regions (sub-step 124), and determine the color value of at least one pixel within each region (sub-step 126); while a second (different) set of one or more machine learning algorithms operate on the color values of the pixel(s) (determined by the first set of one or more machine learning algorithms) to determine the chromophore concentration of the area of skin tissue. In some implementations, the same set of one or machine learning algorithms performs all of the sub-steps of step 120.

In some implementations, the set of machine learning algorithms that determines the chromophore concentration of the area of skin is trained to determine the temporal chromophore signal (e.g., the chromophore concentration for each of a plurality of frames). In some implementations, this set of machine learning algorithms is also trained to apply the one or more filtering operations, to determine the illumination source used when the image data was generated (e.g., to determine one or more characteristics of the illumination source), and to perform other steps. In some implementations, the set of machine learning algorithms that determines the chromophore concentration (a single chromophore concentration measurement or a plurality of chromophore concentration measurements forming the temporal chromophore signal) include a convolutional neural network. In some implementations, this convolutional neural network is configured to receive the color values of pixels representing the area of skin tissue within the image data, and to output a single value of the concentration of the one or more chromophores, and/or a plurality of values of the concentration of the one or more chromophores (e.g., the temporal chromophore signal).

In some implementations, one or more machine learning algorithms are used to determine the value of at least one blood flow metric based on the chromophore concentration measurements. For example, a transformer algorithm can be trained to take the temporal chromophore signal as input, and output one or more blood pressure values.

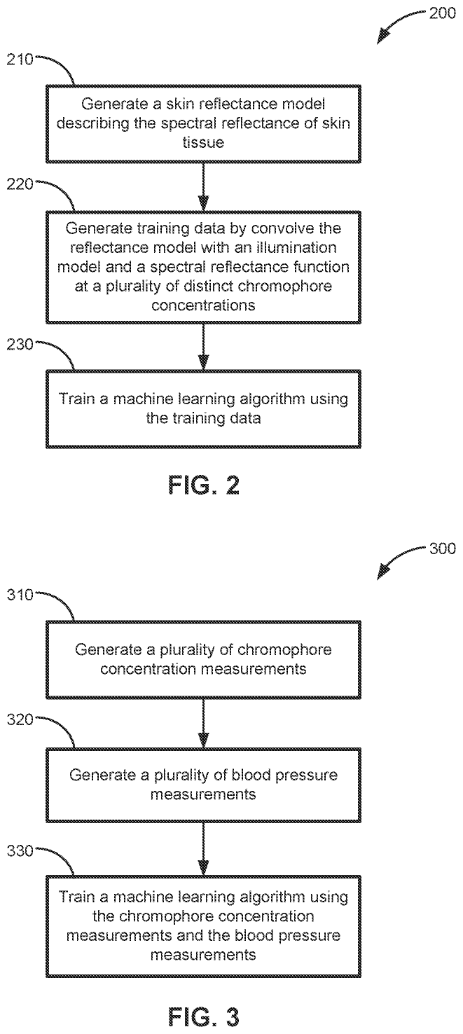

FIG. 2 illustrates a flowchart of a method 200 for training one or more machine learning algorithms. In some implementations, method 200 is used to train the one or more machine learning algorithms that implement step 120 of method 100, where the concentration of the one or more chromophores in the area of skin tissue is determined based on the image data. At step 210, a skin reflectance model is generated that describes the spectral reflectance of skin tissue (e.g., human skin tissue). In some implementations, the skin reflectance model of the skin tissue describes how light that is incident on the skin tissue (e.g., strikes the skin tissue) reflects off of the skin tissue, as a function of the concentration of one or more chromophores in the skin tissue.

In some implementations, the skin reflectance model is formed from a plurality of sub-models combined together, where each sub-model describes how light reflects off of different layers of skin tissue. For example, the skin reflectance model can be formed from a first sub-model that describes how light reflects off of the skin tissue based on the chromophore concentration in a first set of one or more layers of skin tissue, and a second sub-model that describes how light reflects off of the skin tissue based on the chromophore concentration in a second set of one or more layers of the skin tissue. In some implementations, the first set of one or more layers of skin tissue includes an epidermis layer and a dermis layer, and the second set of one or more layers of the skin tissue includes a stratum corneum layer, the epidermis layer, and the dermis layer.

In some implementations, the skin reflectance model is generated by performing at least one Monte Carlo simulation of at least one radiative transport equation for the skin tissue. Different radiative transport equations can be used for different layers or combinations of layers of the skin tissue. In some implementations, the radiative transport equation is given by:

$$( \frac{1}{c} \frac{\partial }{\partial t} + \hat{s} \cdot ∇ + {μ}_{a} ( r ) ) ϕ ( r , \hat{s} , t ) = {μ}_{s} ( r ) {\int }_{{𝕊}^{n - 1}} k ( \hat{s} \cdot {\hat{s}}^{′} ) ϕ ( r , {\hat{s}}^{′} , t ) d {\hat{s}}^{′} + q ( r , {\hat{s}}^{′} , t ) .$$

Here, c is the speed of light; ∂/∂t is the partial derivative operator; ŝ is the unit vector of the direction of light incident on the skin tissue; ∇ is the gradient operator; μₐ(r) is the absorption coefficient of the skin tissue (or the specific layer(s) of skin tissue); ϕ(r, ŝ, t) is the photon density in the skin tissue (or the specific layer(s) of skin tissue) as a function of position, unit vector of incident light, and time; μₛ(r) is the reflection coefficient of the skin tissue (or the specific layer(s) of skin tissue); k(ŝ·ŝ′) is a scattering kernel that describes how light scatters when it is incident on the skin tissue (or the specific layer(s) of skin tissue); and q(r, ŝ′, t) is a function that represents a source of the light that is incident on the skin tissue. On the right-hand side of the equation, the ′ symbol is used to denote the dummy variable for s, as the right-hand side of the equation includes an integral over all space. μₐ(r) and μₛ(r) are both functions of the concentration of one or more chromophores in the skin tissue (or the specific layer(s) of the skin tissue).

The scattering kernel k(ŝ·ŝ′) follows the format of the scattering phase function defined through the inner product of ŝ·ŝ=cos (θ). Using, for example, the Henyey-Greenstein function, the scattering kernel takes the form of

$$k ( \hat{s} \cdot {\hat{s}}^{′} ) = \frac{1 - {g}^{2}}{1 + {g}^{2} - 2 g cos ( θ )} ,$$

where g is the anisotropy factor of the skin tissue (or the specific layer(s) of skin tissue), and g=2π∫₀^{π}cos(θ) k(cos (θ)) sin(θ) dθ. For the Monte Carlo simulations, g is generally greater than or equal to 0.8.

Thus, the radiative transport equation for the skin tissue (and/or for a given layer or layers of the skin tissue) is a partial integro-differential equation as shown above. The left-hand side of the radiative transport equation describes how the density of photons in a unit cube of the skin tissue changes as a function of space, time, and chromophore concentration. The right-hand side of the radiative transport equation describes the summation of all incident light on the skin tissue (or the specific layer(s) of the skin tissue) and how it is scattered through the skin tissue (or the specific layer(s) of the skin tissue) as a function of at least the chromophore concentration. Thus, the radiative transport equation defines the relationship between the density of photons in the skin tissue, the scattering of light that is incident on the skin tissue, and the chromophore concentration in the skin tissue. The radiative transport equation defines the photon density as a function of at least the chromophore concentration, and describes how scattering of light incident on the skin tissue is affected by the chromophore concentration.

In some implementations, different radiative transport equations can be used for different layers or different sets of layers of the skin tissue. In these implementations, a separate Monte Carlo simulation can be performed for each layer or set of layers, and the outputs can be combined to form the skin reflectance model.

The outputs of the Monte Carlo simulations are equations that provide the photon density ϕᵢ within a unit spatial region (e.g., a unit cube or other unit volume of space) of the tissue. For a given path length lⱼ through each layer of the skin tissue in the Monte Carlo simulations, the photon density in the unit spatial region is given by:

$${ϕ}_{i} = \sum {\prod }_{j = 1}^{n} exp \{ {l}_{j} ( {μ}_{a} + {μ}_{s} ( 1 - g ) ) \} .$$

Here, μₐ is the absorption coefficient in the skin tissue (or the specific layer(s) of the skin tissue), μₛ is the scattering coefficient in the skin tissue (or the specific layer(s) of the skin tissue), and j refers to the number of layers within the skin tissue for each Monte Carlo simulation. For K distinct spatial regions, the total reflectance of a particular number of layers of the skin tissue is given by

$${R}_{i} = \frac{{ϕ}_{i}}{ϕ} K ,$$

where ϕ represents the total number of photons in the number of layers. Thus, the total reflectance of the skin tissue as a function of the wavelength of the incident light, R(λ), can be obtained.

In some implementations, the skin reflectance model can include additional reflectances in addition to those obtained by performing Monte Carlo simulations of the radiative transport equations. For example, the diffusion approximation derived using spherical harmonics and the simplified Kubelka-Munk equations,

$$\frac{dl}{dx} = - ( s + k ) I + sJ and \frac{dJ}{dx} = ( s + k ) J - sI ,$$

can be used to form additional reflectances. Here, I and J represent forward and backward travelling light intensities, respectively, and s and k are reduced scattering and absorption coefficients, respectively, for individual layers of skin, determined by chromophore concentrations.

At step 220 of method 200, training data can be generated using the skin reflectance model. Generally, the training data will include a plurality of training data points, where each training data point includes (i) a pixel color value in image data representing the skin tissue, and (ii) a respective known concentration of the one or more chromophores in the skin tissue that corresponds to that specific pixel color value. The training data is obtained by simulating the color of pixels in image data generated by an image sensor that detects light reflected off of skin tissue that has a known chromophore concentration.

In some implementations, the color value of a pixel that corresponds to a specific chromophore concentration is obtained by determining a plurality of fractional pixel color values for each individual wavelength within a range of wavelengths that includes a plurality of wavelengths. Thus, each fractional pixel color value for a known chromophore concentration is associated with a respective one wavelength within the range of wavelengths.

In some implementations, determining the fractional pixel color value for a respective known chromophore concentration (e.g., the pixel color value for the respective known chromophore concentration at a specific wavelength of incident light) includes determining the value of multiple parameters associated with the specific wavelength of light, and multiplying the parameters. A first parameters is a simulated intensity value of incident light on the skin tissue is determined. The first parameter can be obtained using known illumination models that simulated different illumination conditions. These illumination models can include, for example, D50, D55, D60, F2, etc.

A second parameter is a simulated reflectance value of the incident light (e.g., how much of the simulated incident light is reflected off of the skin tissue). The second parameter can be obtained using the skin reflectance model for the respective known chromophore concentration.

A third parameter is the simulated spectral response of an image sensors that detects the reflected incident light. The third parameter can be obtained using known spectral response functions of one or more image sensors that could be used to detect the reflected light. The spectral response function defines how an image sensor converts a detected intensity of light at a specific wavelength into individual pixel color values.

Each of these three parameters is determined for each respective wavelength in the range of wavelengths. A fourth parameter is the difference between successive wavelengths for which the first three parameters are determined. These four parameters for a given wavelength are multiplied together to obtain the fractional pixel color value for the respective known chromophore concentration. Then, all of the fractional pixel color values for the respective known chromophore concentration can be added together to obtain the pixel color value for the respective known chromophore concentration.

The multiplication and summation of these parameters is given by the equation P_{c}=Σᵢ₌₁^{N}I(λᵢ)S_{c}(λᵢ)R(λᵢ)δλ. Here, I(λᵢ) represents the intensity of incident light at a specific wavelength λᵢ, S_{c}(λᵢ) represents the spectral response of an image sensor at wavelength λᵢ, R(λᵢ) represents the reflectance of incident light at the wavelength λᵢ for the respective known chromophore concentration, and δλ represents the difference between successive wavelengths in the wavelength range (e.g., the different between wavelength λₓ and wavelength λₓ₊₁). The multiplication of each of those four parameters for the wavelength λᵢ represents the fractional pixel color value for the wavelength λᵢ, and the sum of the fractional pixel color values is equal to the pixel color value P_{c} for the respective known chromophore concentration. In some implementations, the wavelength range over which these parameters are summed is about 400 nm to about 800 nm. In other implementations, this wavelength range is about 350 nm to about 700 nm.

Thus, the training data is obtained by simulating how an image sensor with a known spectral response function would generate pixel color values if the image sensor detected light that was generated by a known illumination source and reflected off of an area of skin tissue having a known chromophore concentration. By performing this simulation for a plurality of known chromophore concentrations, the training data is obtained.

Finally, at step 230 of method 200, one or more machine learning algorithms are trained using the training data. In some implementations, the one or more machine learning algorithms includes a convolutional neural network (CNN). The CNN is trained to determine chromophore concentrations based on pixel color values input into the CNN. In some implementations, details associated with the simulated illumination also form part of the training data, and are input into the CNN. The one or more machine learning algorithms can be trained using any suitable technique, such as backpropagation and/or stochastic gradient descent. Once the machine learning algorithm is trained, it can be used to determine an unknown concentration of one more chromophores in an area of skin tissue of a subject, based on image data associated with the area of skin tissue of the subject, as performed at step 120 of method 100.

FIG. 3 illustrates a flowchart of a method 300 for training one or more machine learning algorithms. In some implementations, method 300 is used to train the one or more machine learning algorithms that implement step 130 of method 100, wherein the concentration of the one or more chromophores in the area of skin tissue is determined based on the image data. At step 310, a plurality of chromophore concentration measurements is obtained. In some implementations, these chromophore concentration measurements are obtained using one or more image capture devices (such as digital cameras and/or digital video cameras) and a trained machine learning algorithm, such as the machine learning algorithm trained in method 200. Thus, the chromophore concentration measurements can be obtained using method 100, and step 310 of method 300 can in some implementations to be the combination of steps 110 and 120 of method 100.

At step 320, a plurality of blood pressure measurements is obtained. The blood pressure measurements can be obtained using any suitable method, including via the use of a blood pressure cuff or other blood pressure monitor. Generally, the chromophore concentration measurements and the blood pressure measurements are obtained simultaneously. Thus, each individual chromophore concentration measurement (or each distinct plurality of chromophore concentration measurements) is correlated with a single blood pressure measurement. At step 330, a machine learning algorithm is trained using the chromophore concentration measurements and the blood pressure measurements. After the machine learning algorithm has been trained, a chromophore concentration measurement (or a distinct plurality of chromophore concentration measurements) can be input into the machine learning algorithm, which will then output a corresponding blood pressure measurement. In some implementations, the machine learning algorithm is a transformer algorithm.

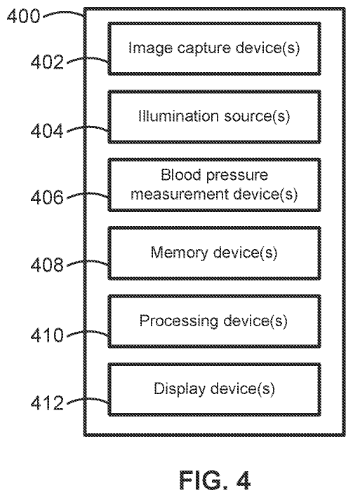

FIG. 4 shows a block diagram of an example system 400 that can be used to implement, wholly or partially, any of methods 100, 200, or 300. The system 400 includes one or more image capture devices 402, one or more illumination sources 404, one or more blood pressure measurement devices 406, one or more memory devices 408, one or more processing devices 410, one or more display devices 412, or any combination thereof. The image capture devices 402 can include digital cameras, digital video cameras, and other types of image sensors, and can be used to generate the image data in step 110 of method 100, and to generate the image data that is used to obtain the chromophore concentration measurements in step 310 of method 300. The one or more illumination sources 404 can include any suitable combination of lights, LEDs, etc., and can be used to illuminate the area of the skin tissue of the subject when the image data is generated in step 110 of method 100, and when the chromophore concentration measurements are obtained in step 310 of method 300.

The one or more blood pressure measurement devices 406 can any include any suitable device, such as a blood pressure cuff or other device. The one or more blood pressure measurement devices 406 can be used to generate the blood pressure measurements in step 320 of method 300. The one or more memory devices 408 can be used to store any data that is used to implement and of methods 100, 200, 300; and/or any data that is generated during the implementation of methods 100, 200, and 300. For example, the one or more memory devices 408 can instructions and data that implement the various machine learning algorithms. The one or more memory devices 408 can also store the various types of image data (real and simulated) that is utilized and/or generated. The one or more processing devices 410 can by any suitable processing device that can execute instructions (such as those stored on the one or more memory devices 408) to implement any of methods 100, 200, 300; to implement the various machine learning algorithms, etc. In some implementations, the memory devices 408 and the processing devices 410 can be formed as part of the same computing system or workstation. In other implementations, the memory devices 408 and the processing devices 410 can be distributed across different physical locations. The one or more display devices 412 can be used to display any type of information associated with the methods 100, 200, and 300, such as chromophore concentration measurements, blood pressure measurements, images and/or video generated from the image data, etc.

In some implementations, methods 100, 200, and/or 300 (and/or any of the various implementations of methods 100, 200, and/or 300 described herein) can be implemented using a system that includes a processing device and a memory. The processing device includes one or more processors. The memory has stored thereon machine-readable instructions. The processing device is coupled to the memory, and methods 100, 200, and/or 300 (and/or any of the various implementations of methods 100, 200, and/or 300 described herein) can be implemented when the machine-readable instructions in the memory are executed by at least one of the one or more processors of the processing device.

Generally, methods 100, 200, and/or 300 (and/or any of the various implementations of methods 100, 200, and/or 300 described herein) can be implemented using a system having a processing device with one or more processors, and a memory storing machine readable instructions. The control system can be coupled to the memory, and methods 100, 200, and/or 300 (and/or any of the various implementations of methods 100, 200, and/or 300 described herein) can be implemented when the machine readable instructions are executed by at least one of the processors of the control system. Methods 100, 200, and/or 300 (and/or any of the various implementations of methods 100, 200, and/or 300 described herein) can also be implemented using a computer program product (such as a non-transitory computer readable medium) comprising instructions that when executed by a computer, cause the computer to carry out the steps of methods 100, 200, and/or 300 (and/or any of the various implementations of methods 100, 200, and/or 300 described herein).

One or more elements or aspects or steps, or any portion(s) thereof, from one or more of any of claims 1-43 below can be combined with one or more elements or aspects or steps, or any portion(s) thereof, from one or more of any of the other claims 1-76 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method for analyzing blood flow in a subject, the method comprising:
generating image data of an area of skin of the subject, the image data reproducible as one or more images of the area of skin of the subject, one or more videos of the area of skin of the subject, or both;
analyzing at least a portion of the image data to determine a concentration of one or more chromophores within the area of skin of the subject; and
determining, based at least in part on the concentration of the one or more chromophores, a value of at least one metric associated with blood flow of the subject.

2. The method of claim 1, wherein the one or more chromophores include hemoglobin, melanin, or both.

3. The method of claim 1, wherein the area of skin of the subject includes at least a portion of a face of the subject.

4. The method of claim 1, wherein analyzing at least the portion of the image data includes inputting at least the portion of the image data into one or more trained machine learning algorithms, the one or more trained machine learning algorithms trained to output an indication of the concentration of the one or more chromophores within the area of the skin of the subject.

5. The method of claim 4, wherein the one or more trained machine learning algorithms are configured to:
identify one or more landmarks within the area of skin of the subject;
based at least in part on the identified landmarks, divide the area of skin of the subject into a plurality of regions; and
determine the concentration of the one or more chromophores of each of the plurality of regions based on a color value of the at least one pixel within each of the plurality of regions.

6. The method of claim 5, wherein the image data is representative of the area of the skin of the subject over a time period and includes a plurality of frames, each of the plurality of frames being reproducible as an image of the area of the skin at a distinct point in time within the time period, and wherein the one or more trained machine learning algorithms are configured to determine the concentration of the one or more chromophores of each of the plurality of regions for each of the plurality of frames within the time period.

7. The method of claim 6, further comprising forming a temporal chromophore signal for the area of the skin of the subject based at least in part on the concentration of the one or more chromophores of each of the plurality of regions at each of the plurality of frames within the time period.

8. The method of claim 7, wherein the temporal chromophore signal represents a spatial variation of the concentration of the one or more chromophores across the area of skin of the subject, and a temporal variation of the concentration of the one or more chromophores across the time period.

9. The method of claim 7, wherein the one or more trained machine learning algorithms are configured to form the temporal chromophore signal.

10. The method of claim 7, further comprising applying one or more filtering operations to the temporal chromophore signal to remove an influence of a cardiac cycle of the subject on the temporal chromophore signal.

11. The method of claim 10, wherein the one or more trained machine learning algorithms are configured to apply the one or more filtering operations.

12. The method of claim 10, wherein the one or more filtering operations include a Butterworth filter, an elliptical filter, a band-pass filter, or any combination thereof.

13. The method of claim 10, wherein the one or more filtering operations are configured to filter out variations in the temporal chromophore signal having a frequency corresponding to a frequency of the cardiac cycle of the subject.

14. The method of claim 13, wherein the frequency of the cardiac cycle of the subject is between about 0.01 Hz and about 5.0 Hz.

15. The method of claim 7, wherein determining the value of at least one metric associated with blood flow of the subject includes determining at least one blood pressure value based at least in part on the concentration of the one or more chromophores for each of the plurality of regions.

16. The method of claim 15, wherein determining the value of at least one metric associated with blood flow of the subject includes determining a time-varying blood pressure signal based at least in part on the temporal chromophore signal.

17. The method of claim 15, wherein the blood pressure of the subject is a mean arterial blood pressure, a systolic blood pressure, a diastolic blood pressure, or any combination thereof.

18. The method of claim 15, wherein the one or more trained machine learning algorithms are configured to determine the at least one blood pressure value.

19. The method of claim 15, wherein one or more additional trained machine learning algorithms are configured to determine the at least one blood pressure value, the one or more additional trained machine learning algorithms that generate the at least one blood pressure value being different than the one or more trained machine learning algorithms that determine the concentration of the one or more chromophores.

20. The method of claim 19, wherein the one or more additional trained machine learning algorithms includes at least one transformer.

21. The method of claim 5, wherein the image data is generated while the area of skin of the subject is illuminated by one or more illumination sources, and wherein the determination of the concentration of the one or more chromophores is based at least in part on one or more characteristics of the one or more illumination sources.

22. The method of claim 21, wherein the one or more trained machine learning algorithms are configured to determine the identity of the one or more illumination sources.

23. The method of claim 4, wherein the one or more trained machine learning algorithms includes one or more convolutional neural networks.

24. The method of claim 1, wherein the metric associated with blood flow of the subject is a blood pressure signal.

25. A method of training one or more machine learning algorithms, the method comprising:
generating a skin reflectance model describing a spectral reflectance of skin tissue;
generating a plurality of training data points using the skin reflectance model, each of the plurality of training data points including (i) a pixel color value and (ii) a respective concentration of one or more chromophores corresponding to the pixel color value; and
training the one or more machine learning algorithms with the training data such that the one or more machine learning algorithms are trained to determine a concentration of one or more chromophores in an area of skin of the subject based at least in part on image data associated with the area of skin of a subject.

26. The method of claim 25, wherein the skin reflectance model of the skin tissue describes how light reflects off of the skin tissue as a function of a concentration of the one or more chromophores in the skin tissue.

27. The method of claim 26, wherein the skin reflectance model includes a first sub-model and a second sub-model, the first sub-model describing how light reflects off of the skin tissue as a function of a concentration of the one or more chromophores in a first one or more layers of the skin tissue, the second sub-model describing how light reflects off of the skin tissue as a function of the concentration of the one or more chromophores in a second one or more layers of the skin tissue.

28. The method of claim 25, wherein generating the skin reflectance model includes performing at least one Monte Carlo simulation of at least one radiative transport equation, the at least one radiative transport equation defining a density of photons within the skin tissue as a function of at least a concentration of one or more chromophores in the skin tissue.

29. The method of claim 28, wherein the at least one radiative transport equation describes how scattering of light incident on the skin tissue is affected by the concentration of the one or more chromophores in the skin tissue.

30. The method of claim 28, wherein the at least one radiative transport equation includes at least one partial integro-differential equation.

31. The method of claim 28, wherein generating the skin reflectance model includes:
performing a first Monte Carlo simulation of a first radiative transport equation corresponding to a first set of one or more layers of the skin tissue;
performing a second Monte Carlo simulation of a second radiative transport equation corresponding to a second set of one or more layers of the skin tissue; and
combining an output of the first Monte Carlo simulation and an output of the second Monte Carlo simulation to form the skin reflectance model.

32. The method of claim 31, wherein the first set of one or more layers of the skin tissue includes an epidermis layer and a dermis layer.

33. The method of claim 31, wherein the first set of one or more layers of the skin tissue includes a stratum corneum layer, an epidermis layer, and a dermis layer.

34. The method of claim 25, wherein generating the plurality of training data points includes determining, based at least in part on the skin reflectance model, a pixel color value for each respective one of a plurality of known concentrations of the one or more chromophores.

35. The method of claim 34, wherein determining the pixel color value for each respective known concentration of the one or more chromophores includes determining a plurality of fractional pixel color values for the respective define concentration of the one or more chromophores, each of the plurality of fractional pixel color values corresponding to a respective one wavelength within a range of wavelengths.

36. The method of claim 35, wherein determining the fractional pixel color value for the respective one wavelength includes:
determining, for the respective one wavelength, a simulated intensity value of incident light on skin tissue;
determining, for the respective one wavelength, a simulated reflectance value of the incident light based at least in part on the skin reflectance model;
determining, for the respective one wavelength, a simulated spectral response value of an image sensor that detects the reflected incident light; and
multiplying, for the respective one wavelength, (i) the intensity value, (ii) the reflectance value, (iii) the spectral response value, and (iv) a difference between successive wavelengths in the wavelength range.

37. The method of claim 36, wherein the determination of the simulated intensity value of the incident light is based on one or more known illumination models.

38. The method of claim 36, wherein the determination of the simulated spectral response value of the reflected incident light is based on one or more known spectral response functions of one or more known image sensors.

39. The method of claim 35, wherein determining the pixel color value for each respective known concentration of the one or more chromophores includes adding together the plurality of fractional pixel color values for the respective known concentration of the one or more chromophores.

40. A method of training one or more machine learning algorithms, the method comprising:
generating a plurality of measurements of a concentration of one or more chromophores in skin tissue, each of the plurality of chromophore concentration measurement corresponding to a respective one of a plurality of subjects;
forming training data by correlating each of the plurality of chromophore concentration measurements with a blood pressure measurement of the respective one of the plurality of subjects; and
training one or more machine learning algorithms using the training data such that the one or more machine learning algorithms are trained to determine a measurement of blood pressure in a subject based at least in part on a measurement of the concentration of the one or more chromophores in skin tissue of the subject.

41. A system for analyzing blood flow, the system comprising:
a processing device including one or more processors; and
a memory having stored thereon machine-readable instructions,
wherein the processing device is coupled to the memory, and the method of claim 1 is implemented when the machine-readable instructions in the memory are executed by at least one of the one or more processors of the processing device.

42. A system for analyzing blood flow, the system including a processing device having one or more processors configured to implement the method of claim 1.

43. A computer program product comprising instructions which, when executed by a computer, cause the computer to carry out the method of claim 1.

44. The computer program product of claim 43 wherein the computer program product is a non-transitory computer readable medium.
